(19) United States
(12) Patent Application Publication
(10) Pub. No.: US 2023/0035871 A1
(43) Pub. Date: Feb. 2, 2023

(54) METHOD, COMPOSITION AND KIT FOR FLUORESCENT QUANTITATIVE PCR, AND USE THEREOF

(71) Applicant: SANSURE BIOTECH INC., Changsha (CN)

(72) Inventors: Lizhong DAI, Changsha (CN); Bozhi JI, Changsha (CN); Deyong TAN, Changsha (CN); Jia LIU, Changsha (CN); Xu FAN, Changsha (CN); Zhongping DENG, Changsha (CN)

(21) Appl. No.: 17/891,356

(22) Filed: Aug. 19, 2022

Related U.S. Application Data
(63) Continuation of application No. PCT/CN2020/120819, filed on Oct. 14, 2020.

(30) Foreign Application Priority Data
Jul. 8, 2020 (CN) 202010649826.6

(51) Int. Cl. C12Q 1/6851; C12Q 1/686; C12Q 1/6876

(52) U.S. Cl. CPC C12Q 1/6851; C12Q 1/686; C12Q 1/6876; C12Q 2600/16; C12Q 2527/107; C12Q 2527/137; C12Q 2545/114

(57) ABSTRACT

The present invention relates to the field of molecular biology detection, more particularly to a method for fluorescent quantitative PCR. The method includes: 1) mixing an upstream and downstream primer pair, a fluorescent probe, and a PCR amplification reagent; and 2) carrying out the fluorescent quantitative PCR, where the fluorescent probe has two quenching groups, in which a first quenching group is located at a 3′ end and a second quenching group is labeled on a T base and is 10-15 nt apart from the first quenching group. Using the method for fluorescent quantitative PCR, background signals in the fluorescent quantitative PCR can be reduced; furthermore, the sensitivity of PCR can be improved, the occurrence of false negatives in detection can be reduced, and the amplification efficiency of the fluorescent quantitative PCR can also be improved.

### CROSS-REFERENCE TO RELATED APPLICATION

This application is a continuation application of International patent application No. PCT/CN2020/120819, filed on Oct. 14, 2020, which claims the benefit and priority of Chinese patent application No. CN202010649826.6, filed on Jul. 8, 2020, each of which is incorporated herein by reference in its entirety and for all purposes.

### TECHNICAL FIELD

The present invention relates to the field of molecular biology detection, and in particular, to the field of fluorescent quantitative PCR detection.

### BACKGROUND

Nucleic acid Polymerase Chain Reaction (PCR) technology is a molecular biology technology used for amplifying specific nucleic acid fragments (DNA/RNA), so that the number of the gene fragments is increased by tens of thousands of times. Its core principle is that a fluorescent probe primer binds and hybridizes with a complementary sequence in a single-stranded DNA template to form a partial double strand, and DNA synthesis is carried out under the action of DNA polymerase. The binding of the fluorescent probe primer to the single-stranded DNA template is based on the principle of base pairing: base pairing follows the Watson-Crick base pairing principle of G (guanine): C (cytosine), A (adenine): T (thymine)/U (uracil). PCR is widely used in medical diagnosis, scientific research, bioengineering, agriculture and other disciplines. Quantitative Real-time PCR (qPCR) is a method for measuring the total quantity of products after each PCR cycle by using fluorescent chemicals in nucleic acid amplification reactions. It is also a method for quantitative analysis of a specific DNA sequence in a to-be-tested sample by means of an internal or external reference method. For qPCR, real-time detection is performed on a PCR process through fluorescent signals during a PCR amplification process. Since there is a linear relationship between the Ct value of the template and the initial copy number of the template in the exponential period of PCR amplification, it becomes the basis for quantification. Fluorescent indicators for qPCR detection are mainly divided into two categories: one is fluorescent probes, such as Taqman probes, and molecular beacon probes; and the other is fluorescent dyes that can bind to double-stranded DNA, such as SYBR Green and Eva Green.

As early as in the 1970s, scientist Korana has proposed the idea of nucleic acid amplification in vitro. In 1983, American scientist Kery Mullis got the inspiration for in vitro nucleic acid amplification when he was studying nucleic acid sequencing methods: using test tubes to simulate the natural process of DNA replication in vivo. By providing a series of suitable conditions including template DNA, fluorescent probe primers, DNA polymerase, suitable buffer system, temperature and time for DNA denaturation, rejuvenation and extension, a DNA molecule with known sequences at both ends can be geometrically amplified. Such technology for in vitro DNA amplification has undergone a series of subsequent optimizations and improvements, and has been successfully applied for the world's first PCR invention patent, U.S. Pat. No. 4,683,202, in 1987.

Since then, with the use and promotion of PCR technology in clinical and scientific research, subsequent patents have continued to enrich and optimize the details of PCR technology. In 1992, Higuchi et al. first proposed the use of a dynamic PCR method and a closed fluorescence collection approach to detect and analyze the number of target genes, i.e., real-time fluorescent quantitative PCR technology. Real-time fluorescent quantitative PCR analysis can be used for quantitative analysis by detecting the quantity of amplified products (product-labeled fluorescence intensity) which is directly related to the quantity of the initial target gene. The cycle number Ct value (Cycle threshold) at which the increased fluorescence signal of the amplified product reaches a logarithmic phase has a negative linear relationship with the logarithm of the initial copy number of the template, that is, the number of amplification cycles required for doubling the initial template dilution to achieve the same logarithmic phase fluorescence intensity is increased by one cycle (Ct). The increased signal of the amplified product can be displayed by a fluorescent dye of the product DNA, such as Sybr Green I, or detected by a fluorescent probe with a quenching group, such as a Taqman probe. Probes with fluorescent quenching groups include fluorescent labeled probes hydrolyzed by Taq enzyme in qPCR technology and MGB probes that increase binding efficiency on the basis of hydrolyzed fluorescent probes. A variety of subsequent hybridization probe types, such as hybridization probes of a stem-loop structure, two-hybridization (FRET) probes and other novel probes have related uses but all have large limitations in the scope of use, and the sensitivity of these probes still needs to be further enhanced.

In addition, in the application of clinical tests, false negatives often occur due to the low sensitivity of the detection method. In particular, the ORF1ab gene of SARS-CoV-2 is easy missed due to the low sensitivity of the detection method because of its low expression level.

Therefore, there is a need in the art for a method and product that can improve the sensitivity of PCR and reduce the occurrence of false negatives.

### SUMMARY

In view of the above, in a first aspect, the present invention provides a method for fluorescent quantitative PCR, the method including:

1) mixing an upstream and downstream primer pair, a fluorescent probe, and a PCR amplification reagent; and

2) carrying out the fluorescent quantitative PCR,

wherein, the fluorescent probe has two quenching groups, in which the first quenching group is located at the 3′ end and the second quenching group is labeled on the T base and is 10-15 nt apart from the first quenching group.

By means of using the method for fluorescent quantitative PCR of the present invention, background signals in the fluorescent quantitative PCR can be reduced, and furthermore, the sensitivity of PCR can be improved, the occurrence of false negatives in detection can be reduced, and the amplification efficiency of the fluorescent quantitative PCR can also be improved.

Without wishing to be bound by theory, a single quenching group does not completely quench the fluorescence of a fluorophore, resulting in a higher background signal. Using the fluorescent probe of the present invention having two quenching groups as defined above further successfully reduces the background signals without affecting the progress of the reaction, thereby improving the sensitivity of PCR, and improving the PCR amplification efficiency.

In preferred embodiments, the method further includes mixing an additional additive, and the additional additive is any one or more of Formamide, SDS, and Proclin antibiotics.

The additional additive of the present invention can further cooperate with the fluorescent probe having two quenching groups, so that the sensitivity is further improved, and the detection result is more accurate.

In some specific embodiments, the additional additive used in the present invention has a pH value between 7.0 and 8.8, and more preferably, the PCR amplification reagent used in the present invention has a pH value of 7.5.

In a specific embodiment, the additional additive is Formamide, SDS and Proclin 300, which has a final concentration of 0.01% to 0.05% (v/v) after being added to a PCR reaction solution.

Furthermore, the fluorescent probe has a length of 18-35 bp, and more preferably, the fluorescent probe has a length of 22-28 bp.

Furthermore, the fluorescent probe has a Tm value of 55-70° C., and more preferably, the fluorescent probe has a Tm value of 65-70° C.

Furthermore, the second fluorescent probe has a GC content not exceeding 60%.

The quenching groups can be selected from BHQ1, BHQ2, and MGB, but are not limited thereto.

In a specific embodiment, the first quenching group and the second quenching group are the same quenching group. Both the first and second quenching groups can be labeled using methods well known to those skilled in the art.

The fluorescent probe also has a fluorescent group, and the fluorescent group can be selected from FAM, HEX, ROX, VIC, CY5, 5-TAMRA, TET, CY3 and JOE, but is not limited thereto.

The “fluorescent quantitative PCR” mentioned in the present invention is a method for measuring the total quantity of products after each polymerase chain reaction (PCR) cycle by using fluorescent chemicals in nucleic acid amplification reactions. It is also a method for quantitative analysis of a specific nucleic acid sequence in a to-be-tested sample by means of an internal or external reference method.

The “PCR amplification reagent” mentioned in the present invention refers to a reagent used for real-time fluorescent quantitative nucleic acid amplification detection. Those skilled in the art can understand that a qPCR amplification reagent usually contains DNA polymerase, dNTP, a PCR buffer and the like. For example, when the detection object is RNA, the reagent may further include reverse transcriptase. It is not difficult to understand that those skilled in the art can determine the composition and concentration of the PCR amplification reagent according to specific needs (such as, the type and content of samples).

In a second aspect, the present invention provides a method for one-step fluorescent quantitative PCR, the method including:

1) mixing a sample releasing agent, an upstream and downstream primer pair, a fluorescent probe, a PCR amplification reagent, and a sample; and

2) carrying out the fluorescent quantitative PCR,

where the fluorescent probe has two quenching groups, in which the first quenching group is located at the 3′ end and the second quenching group is labeled on the T base and is 10-15 nt apart from the first quenching group; and

the method includes no purification and/or extraction steps of nucleic acid.

The “sample releasing agent” mentioned in the present invention refers to a chemical reagent that can release nucleic acid in a sample and can be used for PCR without the need for purification and/or extraction of nucleic acid, such as a strongly acidic or basic chemical reagent. An exemplary sample releasing agent may include one or more of 0.01-0.5 mmol/L of surfactin, 100-200 mmol/L of potassium chloride, 50-200 mmol/L of lithium chloride, triethanolamine lauryl sulfate having a mass/volume ratio of 0.1% to 1%, Nonidet P-40 (NP-40) having a volume/volume ratio of 0.1% to 1%, sodium clodecyl sulfonate having a mass/volume ratio of 0.01% to 2%, ethanol having a volume/volume ratio of 0.05% to 1%, and other components. However, the present invention is not limited thereto.

The “one-step method” mentioned in the present invention refers to the Extraction Free Nucleic Acid Release and Amplification Technology (EFNART). The technology refers to performing direct sample nucleic acid amplification detection directly by means of a sample nucleic acid releasing agent with strong alkaline properties and a highly compatible amplification system without the need for nucleic acid extraction or purification of the sample.

Using the method of the present invention can improve the sensitivity of the “one-step method”, overcome the defects of low sensitivity and inaccurate detection result of the “one-step method”, and avoid the occurrence of false negatives. In addition, using the one-step method also saves time, completes the detection of nucleic acid in a very short time, and improves the detection efficiency (for example, bringing accurate diagnosis results and treatment plans to patients as early as possible opportunity, and playing a key role in hindering the spread of major infectious diseases).

The “sample” mentioned in the present invention refers to a substance containing to-be-tested nucleic acid. The sample may include, but is not limited to, animal and plant cells, bacteria, viruses, fungi, etc., as well as body fluids, tissues, organs, etc. including these.

In particular, the samples mentioned in the present invention are mucus, sputum, pus and urine, etc., and since such samples need to be preserved in a preservation solution, which contains some interfering substances such as antibiotics and surfactants, the sensitivity is usually low when a nucleic acid extraction-free detection method is used.

In a third aspect, the present invention provides a composition for fluorescent quantitative PCR, including an upstream and downstream primer pair, a fluorescent probe, and a PCR amplification reagent;

wherein, the fluorescent probe has two quenching groups, in which the first quenching group is located at the 3′ end and the second quenching group is labeled on the T base and is 10-15 nt apart from the first quenching group.

Furthermore, the composition further includes an additional additive, and the additional additive is any one or more of Formamide, SDS, and Proclin antibiotics.

In some specific embodiments, the additional additive used in the composition has a pH value between 7.5 and 8.8, and more preferably, the PCR amplification reagent used in the present invention has a pH value of 8.5.

In a specific embodiment, the additional additive is SDS and Proclin 300, which has a final concentration of 0.01% to 0.05% (v/v) after being added to a PCR reaction solution.

Furthermore, the fluorescent probe has a length of 18-35 bp, and more preferably, the fluorescent probe has a length of 22-28 bp.

Furthermore, the fluorescent probe has a Tm value of 55-70° C. and more preferably, the fluorescent probe has a Tm value of 55-70° C.

Furthermore, the fluorescent probe has a GC content of not exceeding 60%.

The quenching groups can be selected from BHQ1, BHQ2, and MGB, but are not limited thereto.

In a specific embodiment, the first quenching group and the second quenching group are the same quenching group.

The fluorescent probe also has a fluorescent group, and the fluorescent group can be selected from FAM, HEX, ROX, VIC, CY5, 5-TAMRA, TET, CY3 and JOE, but is not limited thereto.

In a fourth aspect, the present invention provides use of the above-mentioned composition in the preparation of a kit for fluorescent quantitative PCR.

Furthermore, the present invention provides use of the above-mentioned composition in the preparation of a kit for one-step fluorescent quantitative PCR.

In a fifth aspect, the present invention provides a kit for fluorescent quantitative PCR, the kit including the above-mentioned composition.

### BRIEF DESCRIPTION OF THE DRAWINGS

FIG. 1 to FIG. 6 show detection results of the composition of the present invention and the compositions of comparative examples for the detection of SARS-CoV-2.

### DETAILED DESCRIPTION

Hereinafter, the present invention is described in detail with reference to specific embodiments and examples, and the advantages and various effects of the present invention may be more clearly presented therefrom. It should be understood by those skilled in the art that these specific embodiments and examples are intended to illustrate the present invention, instead of limiting the present invention.

### Example 1. The Fluorescent Probe Having Two Quenching Groups of the Present Invention Improves the Sensitivity of PCR

In order to evaluate the application of a double quenching design method of a probe in the present invention, the influence on PCR was investigated. The double quenching design method with the probe of the present invention was compared with a probe having a single quenching group in a PCR amplification reagent. On the premise of not changing the primer sequences and amplification components, the nucleic acid detection of weakly positive samples by different probe designs in the same PCR amplification system was investigated. The comparison method was to use a clinically diagnosed positive low-concentration respiratory syncytial virus (RSV) sample for detection, and the same sample was detected 10 times to verify the positive detection efficiency of the sample. The specific components of each group are shown in Table 1.

TABLE 1
Specific components of different probe
design schemes

| | Amplifi- | Amplifi- | |
|---|---|---|---|
| | cation | cation | Control |
| | scheme 1 of | scheme 2 of | amplifi- |
| | the present | the present | cation |
| | invention | invention | scheme |
| Upstream | GCAAATATGGAAACATACGTGAACA | | |
| primer | (SEQ ID NO: 1) | | |
| sequence | | | |
| Downstream | RGATGAYTGGAACATRGGCACC | | |
| primer | (SEQ ID NO: 2) | | |
| sequence | | | |
| Taqman | 5′-FAM- | 5′-FAM- | 5′-FAM- |
| probe | CAGCWGCTGT | CAGCWGCTGTG | CAGCWGCTGTG |
| sequence | GTAT(T- | TATGT(T- | TATGTGGAGCC |
| | BHQ1)GTGGA | BHQ1)GGAGCC | YTCGTGA-3′- |
| | GCCYTCGTGA- | YTCGTGA-3′- | BHQ1 (SEQ |
| | 3′BHQ1 | BHQ1 (SEQ | ID NO: 5) |
| | (SEQ ID NO: | ID NO: 4) | |
| | 3) | | |

| | | |
|---|---|---|
| Upstream | 10 | pmol |
| primer | | |

dosage

| | | |
|---|---|---|
| Downstream | 10 | pmol |
| primer | | |
| dosage | | |
| Probe | 5 | pmol |
| dosage | | |
| Mg²⁺ | 2 | pmol |
| dNTPs | 2 | pmol |
| Enzyme | 4 | μL |
| mixture | | |
| PCR buffer | 24.65 | μL |
| Sample | 20 | μL |
| dosage | | |
| Total | 50 | μL |
| reaction | | |
| volume | | |

In the above-mentioned comparison experiments, the double quenching design of the probe was investigated, especially the comparison between the design at different base positions and the probe having a single quenching group. The test results show (Table 2) that the probe designs of amplification scheme 1 and amplification scheme 2 of the present invention can significantly improve the detection efficiency for low-concentration RSV samples compared with the conventionally designed comparative amplification scheme without double quenching. However, compared with the amplification method 1/2 of the present invention, although a second quenching group is designed on different bases, there is almost no difference in the influence on the amplification detection efficiency, and there is no significant difference in the detection efficiency of the 10 samples. The experimental results of this group show that for the design of two quenching groups on the same probe, the designs at different positions have no significant difference in detection efficiency.

TABLE 2
Amplification comparison results of different amplification procedures for
10 low-concentration nucleic acid samples

| | | | | | | | | | | |
|---|---|---|---|---|---|---|---|---|---|---|
| Amplification | 35.14 | 35.65 | 34.98 | 36.02 | 36.54 | 3579 | 35.15 | 36.74 | 36.54 | 35.27 |
| scheme 1 of the | | | | | | | | | | |
| present invention | | | | | | | | | | |
| Amplification | 35.15 | 35.31 | 35.69 | 36.46 | 36.93 | 35.82 | 35.25 | 36.47 | 36.75 | 35.46 |
| scheme 2 of the | | | | | | | | | | |
| present invention | | | | | | | | | | |
| Control | 38.84 | 38.18 | 38.12 | NoCt | NoCt | 37.89 | 37.95 | 38.86 | NoCt | 39.12 |
| amplification scheme | | | | | | | | | | |

### Example 2. The Fluorescent Probe Having Two Quenching Groups and Additional Additives of the Present Invention Improve the Sensitivity of PCR

In order to evaluate the application of the double-quenching probe and its PCR amplification reagent components (0.02% of formamide (v/v), 0.03% of SDS (w/v) and 0.01% of Proclin 300 (v/v)) in the present invention, the same probe design principle was employed to compare and modify the fluorescent probe design of respiratory syncytial virus (RSV), and on the premise that the base sequence of the probe remained unchanged, nucleic acid detection of weakly positive samples in the same PCR amplification system was performed using the double quenching group scheme of the present invention and the conventional probe design (5′-FAM specific probe sequence-3′-BHQ1). The comparison method was step-by-step gradient dilution of a clinically diagnosed positive RSV sample: 10-fold dilution (1:9, v/v), 100-fold dilution (1:99, v/v), and 1,000-fold dilution (1:999, v/v), and 10,000-fold dilution (1:9,999, v/v). The detection method was a nucleic acid extraction-free direct amplification detection method for the sample. The method adopted was direct amplification for the sample in sample: sample releasing agent: qPCR reaction solution =10:10:30 (v/v/v) with a total volume of 50 μL. The amplification procedure is shown in Table 3. The specific components of each group are shown in Table 4.

TABLE 3
Amplification detection procedure for nucleic
acid adopted in the present invention

| Step | Temperature | Time | Number of cycles |
|---|---|---|---|
| Reverse transcription | 50° C. | 30 min | 1 |
| Pre-denaturation | 95° C. | 1 min | 1 |
| Denaturation | 95° C. | 15 sec | 40-45 |
| Annealing, extension and | 60° C. | 30 sec | |
| fluorescence collection | | | |

TABLE 4
Specific components of different probe
design schemes

| | Amplification | |
|---|---|---|
| | scheme of the | |
| | present invention | Control scheme |
| Upstream | GCAAATATGGAAACATA | GCAAATATGGAAAC |
| primer | CGTGAACA (SEQ ID | ATACGTGAACA |
| sequence | NO: 1) | (SEQ ID NO: 1) |
| Downstream | RGATGAYTGGAACATRG | RGATGAYTGGAACA |
| primer | GCACC (SEQ ID NO: | TRGGCACC (SEQ |
| sequence | 2) | ID NO: 2) |
| Taqman | 5′-FAM- | 5′-FAM- |
| probe | CAGCWGCTGTGTAT(T- | CAGCWGCTGTGTAT |
| sequence | BHQ1)GTGGAGCCYTCG | GTGGAGCCYTCGTG |
| | TGA-3′-BHQ1 (SEQ | A-3′-BHQ1 (SEQ |
| | ID NO: 3) | ID NO: 5) |

| | | |
|---|---|---|
| Upstream | 10 | pmol |
| primer | | |
| dosage | | |
| Downstream | 10 | pmol |
| primer | | |
| dosage | | |
| Probe | 5 | pmol |
| dosage | | |
| Mg²⁺ | 2 | pmol |
| dNTPs | 2 | pmol |
| Enzyme | 4 | μL |
| mixture | | |

| | | |
|---|---|---|
| Additive | 0.03% (v/v) | 0 |
| group | | |
| concen- | | |
| tration | | |
| PCR buffer | 24.65 μL | 24.65 μL |

| | | |
|---|---|---|
| Sample | 20 | μL |
| dosage | | |
| Total | 50 | μL |
| reaction | | |
| volume | | |

The comparison results in Table 5 show that for the additive components in the PCR amplification system of the present invention, the sensitivity of the extraction-free amplification of the RSV sample is significantly improved. On the premise that the primer probe sequence and the main components of the PCR amplification system remain unchanged, the optimization of the quenching modification scheme of the probe greatly improves the nucleic acid detection capability.

TABLE 5
Sensitivity comparison detection scheme
based on gradient dilution of the sample.

| | Design scheme with | Conventional |
|---|---|---|
| | the probe of the | fluorescent |
| | present invention | probe design scheme |
| | RSV sample | RSV sample |
| | detection Ct value | detection Ct value |
| 10-fold dilution | 29.64 | 31.89 |
| of the sample | | |
| 100-fold dilution | 32.89 | 35.32 |
| of the sample | | |
| 1,000-fold dilution | 36.07 | 38.03 |
| of the sample | | |
| 10,000-fold dilution | 39.46 | No Ct |
| of the sample | | |

### Example 3. The Fluorescent Probe Having Two Quenching Groups and Additional Additives of the Present Invention Improve the Sensitivity of PCR

In order to evaluate the primer probe design scheme and the application of additive components (formamide, SDS and Proclin 300) in the present invention, comparative analysis was performed on a reagent component with the primer probe and PCR additive of the present invention and the conventional 3′-end single quenching group (BHQ1) without the primer probe scheme of the present invention. The comparison method was to use three SARS-CoV-2 nucleic acid samples (sample 01, sample 02, and sample 03) that were clinically diagnosed as positive to conduct a comparative test in the form of 45 μL of a PCR reaction solution +5μL of a nucleic acid sample. The real-time fluorescent quantitative PCR amplification detection procedure is shown in Table 3. The specific components of each group are shown in Table 6.

TABLE 6
Specific components of different probe
design schemes

| | Amplification | |
|---|---|---|
| | scheme of the | |
| | present invention | Control scheme |
| ORF lab | ACAATGCGTTAGCTTACTA | ACAATGCGTTAGCTT |
| upstream | CAAC (SEQ ID NO: 6) | ACTACAAC (SEQ |
| primer | | ID NO: 6) |
| sequence | | |
| ORF lab | TCTAGCCCATTTCAAATCC | TCTAGCCCATTTCAA |
| downstream | TG (SEQ ID NO: 7) | ATCCTG (SEQ ID |
| primer | | NO: 7) |
| sequence | | |
| ORF lab | FAM-TAGGTTTGTACT(T- | TAGGTTTGTACTTGC |
| fluorescent | BHQ1)GCATTGTTATCCG- | ATTGTTATCCG |
| probe | BHQI (SEQ ID NO: 8) | (SEQ ID NO: 9) |
| sequence | | |

| N gene | AGTCCAGATGACCAAATTGGC | |
|---|---|---|
| upstream | (SEQ ID NO: 10) | |
| primer | | |
| sequence | | |
| N gene | ACTGAGATCTTTCATTTTACCGTCAC | |
| downstream | (SEQ ID NO: 11) | |
| primer | | |
| sequence | | |
| N gene | ACTACCGAAGAGCTACCAGACGA | |
| fluorescent | (SEQ ID NO: 12) | |
| probe | | |
| sequence | | |
| Upstream | 10 | pmol |
| primer | | |
| dosage | | |
| Downstream | 10 | pmol |
| primer | | |
| dosage | | |
| Probe | 5 | pmol |
| dosage | | |
| Mg²⁺ | 2 | pmol |
| dNTPs | 2 | pmol |
| Enzyme | 4 | μL |
| mixture | | |

| | | |
|---|---|---|
| Additive | 0.03% (v/v) | 0 |
| group | | |
| concen- | | |
| tration | | |
| PCR buffer | 33.65 μL | 33.65 μL |

| | | |
|---|---|---|
| Nucleic | 5 | μL |
| acid | | |
| dosage | | |
| Total | 50 | μL |
| reaction | | |
| volume | | |

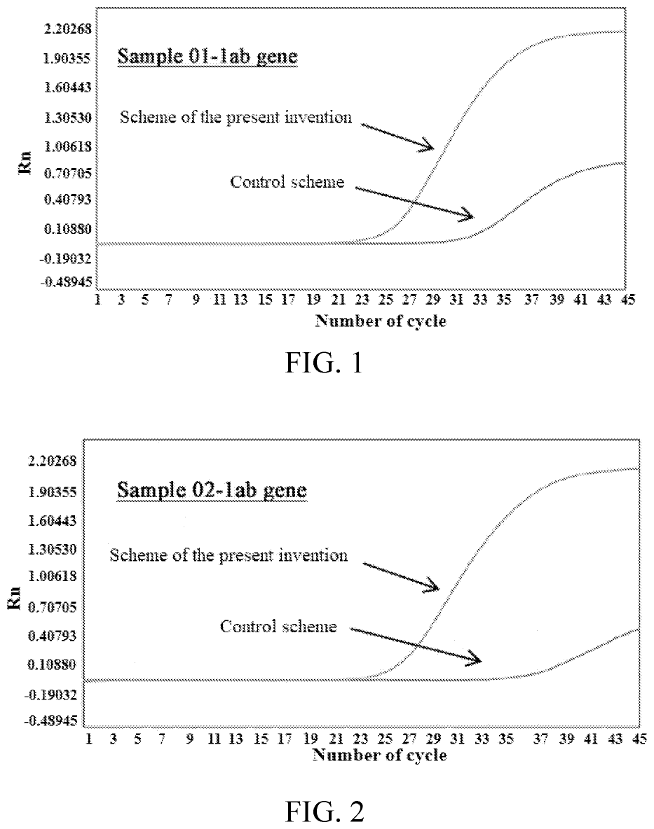

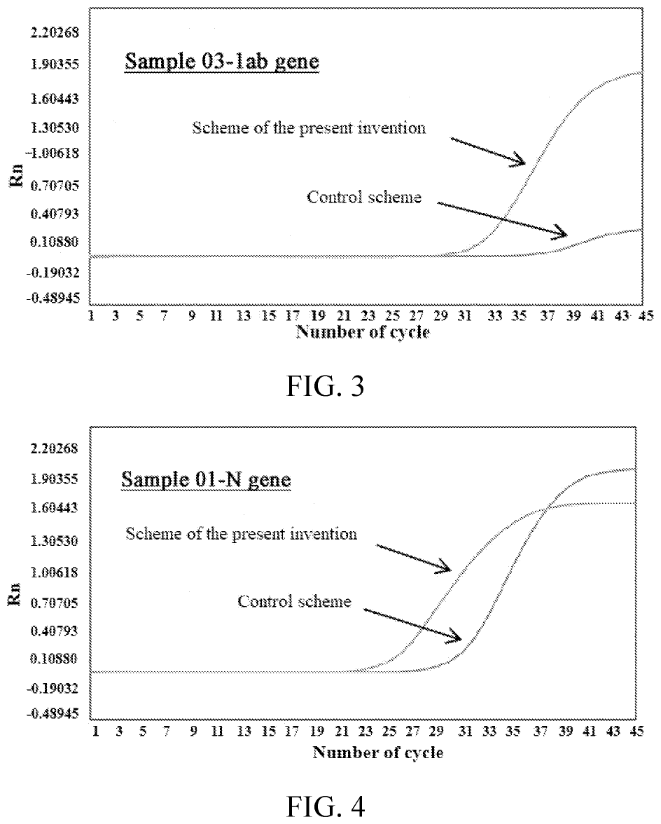

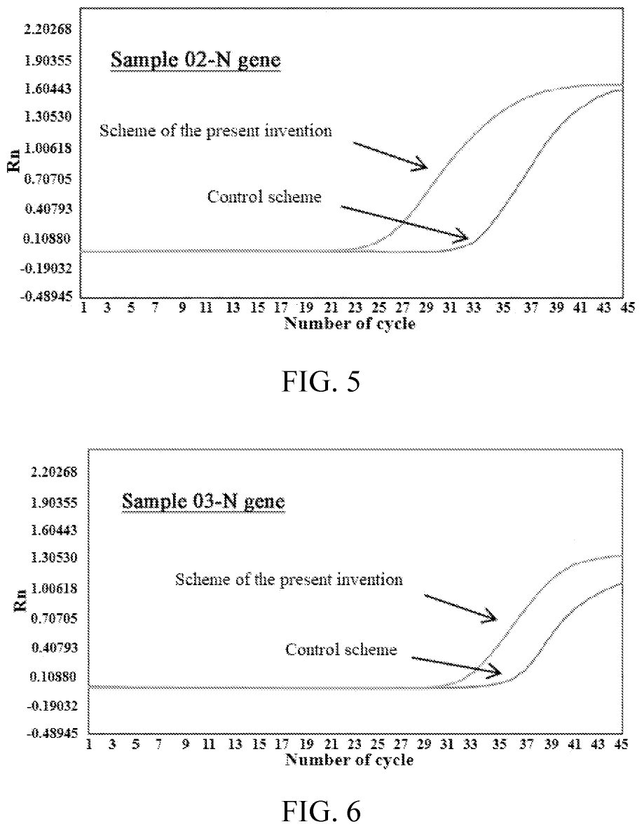

From the comparison results of amplification in FIG. 1 to FIG. 6 and Table 7, on the premise of the same nucleic acid template dosage and primer probe dosage, just because of the change of the probe labeling mode and the necessary additive components, between the two, especially the amplification efficiency of the ORF 1ab gene based on FAM channel is significantly improved. Because the probe modification method of the present invention has obvious advantages in improving the detection sensitivity of nucleic acid reagents.

TABLE 7
Comparison results of the amplification effects of the scheme of the
present invention and the control scheme on three nucleic acid samples

| | PCR reaction solution with | | Commercial PCR | |
|---|---|---|---|---|
| | additives of the present invention | | reaction solution | |
| | ORF lab gene | N gene | ORF lab gene | N gene |
| Sample 01 | 25.05 | 25.20 | 32.92 | 30.10 |
| Sample 02 | 25.35 | 25.45 | 37.91 | 33.17 |
| Sample 03 | 31.88 | 32.19 | 39.69 | 35.24 |

The comparison results in Table 7 show that for the probe modification and additive components in the present invention, the sensitivity of PCR is significantly improved. The detection capability of a kit is negatively correlated with the Cycle threshold (Ct) value, that is, at the same concentration, the smaller the Ct value is, the higher the detection capability is; the larger the Ct value is, the lower the detection capability is; and No Ct means no amplification. The probe modification and additives in the present invention can significantly improve the nucleic acid detection capability, but for different samples, there are differences between samples in the improvement effect of the PCR additives.

### Example 4. The Fluorescent Probe Having Two Quenching Groups and Additional Additives of the Present Invention Improve the Sensitivity of One-Step PCR

In the process of clinical examination application, respiratory samples are highly infectious, such as SARS-CoV-2, SARS, MERS coronavirus, etc., as well as common influenza A and B viruses, all of which are highly pathogenic. Therefore, completing virus detection in a very short time can bring diagnosis results and treatment plans to patients as early as possible. The scheme of the double-quenching probe and the matched PCR amplification additive components in the present invention can also be used in the application of rapid sample detection. The main features of the procedure include two major aspects: the two are both directly amplifying the samples without nucleic acid processing, and are respectively: 1. on the premise of no nucleic acid extraction and purification, directly inactivating the collected samples by using a sample releasing agent with an inactivation function, and releasing the nucleic acid in the virus, and using the relevant PCR amplification reagent components for direct amplification detection; and 2. adopting a reverse transcription procedure and a PCR amplification procedure in a very short time to complete the detection of the virus and giving the detection result within 15-35 minutes. The detection method was a nucleic acid extraction-free direct amplification detection method for the sample. The method adopted was direct amplification for the sample in sample: sample releasing agent: qPCR reaction solution =10:10:30 (v/v/v) with a total volume of 50 μL. In order to compare the detection effects in rapid detection and conventional detection, this experimental scheme adopted the reaction conditions of the present invention under the rapid amplification procedure and the conventional amplification procedure, as well as the conventional probe design scheme under the conventional amplification procedure to compare the detection efficiency of low-concentration nucleic acid samples and negative samples of SARS-CoV-2.

TABLE 8
One-step PCR and conventional PCR in the scheme of the present invention

| | Conventional amplification | | | Rapid amplification | | |
|---|---|---|---|---|---|---|
| | procedure | | | procedure | | |
| | | | Number | | | Number |
| Step | Temperature | Time | of cycles | Temperature | Time | of cycles |
| Reverse | 50° C. | 30 min | 1 | 50° C. | 5 min | 1 |
| transcription | | | | | | |
| Pre-denaturation | 95° C. | 1 min | 1 | 95° C. | 10 sec | 1 |
| Denaturation | 95° C. | 15 sec | 40-45 | 95° C. | 1 sec | 40-45 |
| Annealing, | 60° C. | 30 sec | | 60° C. | 1 sec | |
| extension and | | | | | | |
| fluorescence collection | | | | | | |

TABLE 9
Amplification comparison results of different amplification procedures for
10 low-concentration nucleic acid samples

| | | | | | | | | | | |
|---|---|---|---|---|---|---|---|---|---|---|
| Scheme of the | 32.42 | 32.46 | 32.59 | 31.76 | 32.77 | 32.92 | 31.83 | 32.74 | 32.09 | 32.40 |
| present invention | | | | | | | | | | |
| Conventional | | | | | | | | | | |
| amplification | | | | | | | | | | |
| Scheme of the | 33.15 | 32.84 | 32.16 | 32.56 | 32.98 | 33.23 | 33.75 | 32.47 | 32.95 | 32.63 |
| present invention | | | | | | | | | | |
| Rapid | | | | | | | | | | |
| amplification | | | | | | | | | | |
| Control scheme | 35.84 | 36.18 | 36.28 | 35.98 | 36.32 | 35.45 | 36.74 | 36.12 | 36.29 | 36.83 |
| Conventional | | | | | | | | | | |
| amplification | | | | | | | | | | |
| Control scheme | 38.84 | 39.18 | NoCt | NoCt | NoCt | NoCt | 38.74 | 39.42 | NoCt | NoCt |
| Rapid | | | | | | | | | | |
| amplification | | | | | | | | | | |

As can be seen from the detection results of 10 low-concentration samples, all the low-concentration samples were detected using the scheme of the present invention. In addition, the detection result obtained using the rapid amplification solution was no different from that using the conventional amplification procedure. Therefore, the experimental results prove that the amplification detection scheme based on the double-quenching probe design can be used in both conventional qPCR amplification procedure and rapid qPCR amplification procedure, and there is no significant difference between the two different amplification procedures. The scheme of the present invention can be used for rapid detection of viruses. In the control scheme, the probe design and combination scheme without the conventional single quenching group of the present invention were adopted, and in the conventional amplification procedure, all 10 samples were detected, but the amplified Ct values were delayed. However, in the rapid amplification procedure, only four out of the 10 samples were detected to be positive, thus the detection capability was obviously low. Therefore, the comparison results show that the scheme of the present invention can be used for rapid nucleic acid diagnosis of RNA viruses.

### Example 5. The Fluorescent Probe Having Two Quenching Groups and Additional Additives of the Present Invention Improve the Sensitivity of PCR

In order to evaluate the application of the double-quenching probe and its PCR amplification reagent components (Formamide, SDS and Proclin 300) in the present invention, the same probe design principle was employed to compare and modify the fluorescent probe design of respiratory adenovirus (ADV), and on the premise that the base sequence of the probe remained unchanged, nucleic acid detection of weakly positive samples in the same PCR amplification system was performed by comparing the amplification schemes of the double quenching group scheme of the present invention and the control scheme.

The designs of the four schemes are (as shown in Table 11):

the scheme of the present invention: an amplification reagent containing a double-quenching fluorescent probe and additive components

control scheme 1: a conventional amplification reagent only containing a double-quenching fluorescent probe

control scheme 2: an amplification reagent containing a single-quenching fluorescent probe and an additive

control scheme 3: a conventional amplification reagent only containing a single-quenching fluorescent probe

The comparison method was step-by-step gradient dilution of a clinically diagnosed positive ADV sample: 10-fold dilution (1:9, v/v), 100-fold dilution (1:99, v/v), and 1,000-fold dilution (1:999, v/v). The detection method was a nucleic acid extraction-free direct amplification detection method for the sample. The method adopted was direct amplification for the sample in sample: sample releasing agent: qPCR reaction solution =10:10:30 (v/v/v) with a total volume of 50 μL. The amplification procedure is shown in Table 10.

TABLE 10
Amplification detection procedure for nucleic acid adopted in the present invention.

| | Conventional amplification | | | Rapid amplification | | |
|---|---|---|---|---|---|---|
| | procedure | | | procedure | | |
| | | | Number of | | | Number of |
| Step | Temperature | Time | cycles | Temperature | Time | cycles |
| Pre-denaturation | 95° C. | 1 min | 1 | 95° C. | 10 sec | 1 |
| Denaturation | 95° C. | 15 sec | 40-45 | 95° C. | 3 sec | 40-45 |
| Annealing, extension | 60° C. | 30 sec | | 60° C. | 5 sec | |
| and fluorescence collection | | | | | | |

TABLE 11
Specific components of different probe
design schemes

| | | | |
|---|---|---|---|
| Scheme of | | | |
| the present | Control | Control | Control |
| invention | scheme 1 | scheme 2 | scheme 3 |

| Upstream | TGRAARAGGTAGTTGAGBGTGG (5′-3′) | |
|---|---|---|
| primer sequence | (SEQ ID NO: 13) | |
| Downstream | GYAACKCCCAGTTTTTCAACTTYG (5′-3′) | |
| primer sequence | (SEQ ID NO: 14) | |
| Probe sequence | 5′-FAM- | 5′-FAM- |
| | CYGTGGAYTTCT | CYGTGGAYTT |
| | ACGA(BHQ1)RG | CTACGARGCC |
| | CCATGGA-3′- | ATGGA-3′- |
| | BHQ1 (SEQ ID | BHQ1( SEQ |
| | NO: 15) | ID NO: 15) |

| | | |
|---|---|---|
| Upstream | 10 | pmol |
| primer dosage | | |
| Downstream | 10 | pmol |
| primer dosage | | |
| Probe dosage | 5 | pmol |
| Mg²⁺ | 2 | pmol |
| dNTPs | 2 | pmol |
| Enzyme mixture | 4 | μL |

| | | | | |
|---|---|---|---|---|
| Additive group | 0.03% | 0 | 0.03% | 0 |
| concentration | (v/v) | | (v/v) | |

| | | |
|---|---|---|
| PCR buffer | 24.65 | μL |
| Sample dosage | 20 | μL |
| Total reaction | 50 | μL |
| volume | | |

The comparison results in Table 12 show that there is a significant improvement in the sensitivity of extraction-free amplification of the ADV sample, and the amplification efficiency of the present invention is the best and obviously superior to the schemes based on conventional fluorescent probe design and conventional PCR amplification reagent components.

In addition, the comparison results in Table 12 also prove that the optimization effect of the additives on the two quenching groups is significantly better than that on the single quenching group. In the double quenching group system, the increase of the Ct value by the additive is greater than 1 (having an order of magnitude difference, a difference of 1 in Ct value is equivalent to a difference of one time the template amount), and in the single quenching group system, the increase of the Ct value by the additive is less than 0.5 (essentially equivalent to no increase).

TABLE 12
Sensitivity comparison detection solution
based on gradient dilution of the sample

| | Scheme of the | Control | Control | Control |
|---|---|---|---|---|
| | present | scheme | scheme | scheme |
| | invention | 1 | 2 | 3 |
| Conventional | ADV detection Ct value | | | |
| qPCR procedure | | | | |
| Sample | 29.64 | 30.89 | 32.14 | 32.65 |
| Rapid amplifi- | ADV detection Ct value | | | |
| cation procedure | | | | |
| Sample | 29.76 | 30.93 | 32.65 | 33.02 |

### REFERENCE TO AN ELECTRONIC SEQUENCE LISTING

The contents of the electronic sequence listing (CU692SequenceListing.xml; Size: 14,136 bytes; and Date of Creation: Aug. 17, 2022) is herein incorporated by reference in its entirety.

1. A method for fluorescent quantitative PCR, comprising:
1) mixing an upstream and downstream primer pair, a fluorescent probe, and a PCR amplification reagent; and
2) carrying out the fluorescent quantitative PCR,
wherein the fluorescent probe has two quenching groups, in which a first quenching group is located at a 3′ end and a second quenching group is labeled on a T base and is 10-15 nt apart from the first quenching group.

2. The method according to claim 1, wherein an additional additive is also mixed in step 1) of the method, and the additional additive is any one or more of formamide, SDS, and Proclin antibiotics.

3. The method according to claim 2, wherein the additional additive is SDS and Proclin 300, which has a final concentration of 0.01% to 0.05% (v/v) after being added to a PCR reaction solution.

4. The method according to claim 2, wherein the additional additive has a pH value between 7.0 and 8.8.

5. The method according to claim 1, wherein the fluorescent probe has a length of 18-35 bp.

6. The method according to claim 1, wherein the fluorescent probe has a Tm value of 55-70° C.

7. The method according to claim 1, wherein the fluorescent probe has a GC content not exceeding 60%.

8. The method according to claim 1, wherein the PCR amplification reagent comprises DNA polymerase, dNTP, and a PCR buffer.

9. The method according to claim 1, wherein the first quenching group and the second quenching group are the same quenching group.

10. A method for one-step fluorescent quantitative PCR, comprising:
1) mixing a sample releasing agent, an upstream and downstream primer pair, a fluorescent probe, a PCR amplification reagent, and a sample; and
2) carrying out the fluorescent quantitative PCR,
wherein the fluorescent probe has two quenching groups, in which a first quenching group is located at a 3′ end and a second quenching group is labeled on a T base and is 10-15 nt apart from the first quenching group; and
the method comprises no purification and/or extraction steps of nucleic acid.

11. A composition for fluorescent quantitative PCR, comprising an upstream and downstream primer pair, a fluorescent probe, and a PCR amplification reagent,
wherein the fluorescent probe has two quenching groups, in which a first quenching group is located at a 3′ end and a second quenching group is labeled on a T base and is 10-15 nt apart from the first quenching group.

12. The composition according to claim 11, wherein the composition further comprises an additional additive, and the additional additive is any one or more of formamide, SDS, and Proclin antibiotics.

13. The composition according to claim 12, wherein the additional additive is SDS and Proclin 300, which has a final concentration of 0.01% to 0.05% (v/v) after being added to a PCR reaction solution.

14. The composition according to claim 12, wherein the additional additive has a pH value between 7.0 and 8.8.

15. The composition according to claim 11, wherein the fluorescent probe has a length of 18-35 bp.

16. The composition according to claim 11, wherein the fluorescent probe has a Tm value of 55-70° C.

17. The composition according to claim 11, wherein the fluorescent probe has a GC content not exceeding 60%.

18. The composition according to claim 11, wherein the PCR amplification reagent comprises DNA polymerase, dNTP, and a PCR buffer.

19. The composition according to claim 11, wherein the first quenching group and the second quenching group are the same quenching group.

20. A kit for fluorescent quantitative PCR, wherein the kit comprises the composition according to claim 11.


SEQUENCE LISTING (15 sequences)

SEQ ID NO: 1 - DNA, 25 bp, synthetic construct
  source 1..25: mol_type = other DNA
gcaaatatggaaacatacgtgaaca

SEQ ID NO: 2 - DNA, 22 bp, synthetic construct
  source 1..22: mol_type = other DNA
rgatgaytggaacatrggcacc

SEQ ID NO: 3 - DNA, 30 bp, synthetic construct
  source 1..30: mol_type = other DNA
cagcwgctgtgtattgtggagccytcgtga

SEQ ID NO: 4 - DNA, 30 bp, synthetic construct
  source 1..30: mol_type = other DNA
cagcwgctgtgtatgttggagccytcgtga

SEQ ID NO: 5 - DNA, 29 bp, synthetic construct
  source 1..29: mol_type = other DNA
cagcwgctgtgtatgtggagccytcgtga

SEQ ID NO: 6 - DNA, 23 bp, synthetic construct
  source 1..23: mol_type = other DNA
acaatgcgttagcttactacaac

SEQ ID NO: 7 - DNA, 21 bp, synthetic construct
  source 1..21: mol_type = other DNA
tctagcccatttcaaatcctg

SEQ ID NO: 8 - DNA, 26 bp, synthetic construct
  source 1..26: mol_type = other DNA
taggtttgtacttgcattgttatccg

SEQ ID NO: 9 - DNA, 26 bp, synthetic construct
  source 1..26: mol_type = other DNA
taggtttgtacttgcattgttatccg

SEQ ID NO: 10 - DNA, 21 bp, synthetic construct
  source 1..21: mol_type = other DNA
agtccagatgaccaaattggc

SEQ ID NO: 11 - DNA, 26 bp, synthetic construct
  source 1..26: mol_type = other DNA
actgagatctttcattttaccgtcac

SEQ ID NO: 12 - DNA, 23 bp, synthetic construct
  source 1..23: mol_type = other DNA
actaccgaagagctaccagacga

SEQ ID NO: 13 - DNA, 22 bp, synthetic construct
  source 1..22: mol_type = other DNA
tgraaraggtagttgagbgtgg

SEQ ID NO: 14 - DNA, 24 bp, synthetic construct
  source 1..24: mol_type = other DNA
gyaackcccagtttttcaacttyg

SEQ ID NO: 15 - DNA, 25 bp, synthetic construct
  source 1..25: mol_type = other DNA
cygtggayttctacgargccatgga